(19) United States
(12) Patent Application Publication
(10) Pub. No.: US 2019/0336443 A1
(43) Pub. Date: Nov. 7, 2019

(54) Ready-To-Use Liquid Parenteral Formulations Of Ribavirin

(71) Applicant: Navinta III Inc, Boca Raton, FL (US)

(72) Inventor: Sandipkumar Arvindbhai Patel, Lawrenceville, NJ (US)

(21) Appl. No.: 16/399,288

(22) Filed: Apr. 30, 2019

Related U.S. Application Data
(60) Provisional application No. 62/666,317, filed on May 3, 2018.

(51) Int. Cl. A61K 9/00; A61K 31/7056

(52) U.S. Cl. CPC A61K 9/0078; A61K 31/7056

(57) ABSTRACT

Stable ready to use pharmaceutical compositions of Ribavirin including a primary packaging container containing a sterile aqueous Ribavirin solution having a concentration of 10 mg/mL to 40 mg/mL Ribavirin and a seal sealing the primary packaging container. The Ribavirin solution is free of viable microbial contamination in in accordance with the test methods in 40 USP <71> using Trypticase Soy Broth and Fluid Thioglycollate Medium. A method of manufacturing ready to use Ribavirin solutions by terminally sterilizing a Ribavirin solution. Methods of treating respiratory syncytial virus by administering ready to use 20 mg/mL Ribavirin solutions in a nebulizer.

### FIELD OF THE INVENTION

The present invention relates to parenteral, more preferably inhalation, aqueous formulations of ribavirin.

### BACKGROUND OF THE INVENTION

Ribavirin is a synthetic nucleoside with antiviral activity. Ribavirin compositions have been approved in the United States for the treatment of hepatitis C virus and respiratory syncytial virus. The active ingredient, Ribavirin, is 1-beta-D-ribofuranosyl-1H-1,2,4-triazole-3-carboxamide, with the following structural formula:

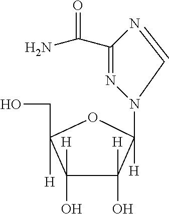

It is a white crystalline compound with a maximum solubility in water of 142 mg/mL at 25° C. and with only a slight solubility in ethanol. The empirical formula is C₈H₁₂N₄O₅ and the molecular weight is 244.21. Ribavirin is available for oral administration to treat chronic hepatitis C infection. Various tablet and capsule formulations are available on the market and an oral solution is marketed by Schering.

A prime concern with any liquid formulation is the stability of the active ingredient, both short term and over time. In general, drug substances are less stable in aqueous media than in the solid dosage form. Thus, it is important to properly stabilize and preserve those formulations, especially if the formulation contains water. Chemical reactions can take place in these products that may involve ingredient-ingredient interaction. Another potential reaction is a container-product interaction that may alter pH, and thus, if the active ingredient is pH sensitive, instability in the form of precipitates or degradation products could result. Ribavirin is one such active ingredient that is pH sensitive, and it is readily degraded by hydrolysis when in a liquid formulation.

The oral solution contains 40 mg/mL Ribavirin and sodium citrate, citric acid, sodium benzoate, glycerol, sucrose, sorbitol liquid (crystallising), propylene glycol, purified water and natural and artificial flavoring. The oral solution shelf life is 3 years, but after opening the product should be used within one month.

A solution pH that promotes hydrolysis will affect ribavirin degradation directly by increasing the amount of ribavirin hydrolysis. Additionally, ribavirin stability is compromised when there is an increase in the formation of reducing agents in the formulation, i.e., the sugar sucrose can be hydrolyzed to the reducing sugars fructose and glucose. These sugars can in turn increase the hydrolysis of ribavirin.

WO 2003037312 proposes alternative liquid syrup formulations. Due to the typically high sugar content in syrups, syrups are susceptible to microbial infestation. Syrups frequently must contain antimicrobial components to ensure safe storage without the proliferation of pathogenic molds, yeasts, bacteria and the like; a typical antimicrobial deemed suitable for use in foods and other ingestible substances is sodium benzoate. The application provides a liquid formulation comprising ribavirin, a buffering system, wherein the pH of the liquid formulation is in the range of about 4.8 to about 5.3, at least one pharmaceutically acceptable sweetening agent, and at least one pharmaceutically acceptable viscosity increasing agent. WO2003037312 teaches it is preferable to increase the non-aqueous proportion of the formulation because it is believed that a decrease in the non-aqueous portion of the formulation will increase the non-polar character of the formulation, thus decreasing ribavirin susceptibility to hydrolysis. The alternative oral formulations have better stability but are susceptible to microbial growth and require preservatives. Moreover, they are not sterile or aseptically manufactured, thus rendering them unusable for parenteral routes of administration.

Due to the hydrolysis concerns, Ribavirin for Inhalation Solution, USP is a sterile, lyophilized powder to be reconstituted for aerosol administration. Each 100-mL glass vial contains 6 grams of ribavirin, and when reconstituted and further diluted to the recommended volume of 300 mL with Sterile Water for Injection, USP or Sterile Water for Inhalation (no preservatives added), will contain 20 mg of ribavirin per mL. The pH is approximately 5.5. Aerosolization is to be carried out in a Small Particle Aerosol Generator (SPAG®-2) nebulizer only. The inhalation solution is indicated for the treatment of hospitalized infants and young children with severe lower respiratory infections due to respiratory syncytial virus.

As per the product label information of VIRAZOLE® Inhalation Solution, Ribavirin has demonstrated significant teratogenic and/or embryocidal potential in all animal species in which adequate studies have been conducted (rodents and rabbits). Therefore, although clinical studies have not been performed, it should be assumed that Ribavirin may cause fetal harm in humans. As a result, medical staff involved in reconstitution and dilution of Ribavirin inhalation solution need to take substantial precautions to avoid exposure to this solution.

Per FDA guidelines, all inhalation solutions must be sterile. The product label information of VIRAZOLE® Inhalation Solution states that “The solution should be inspected visually for particulate matter and discoloration prior to administration. Solutions that have been placed in the SPAG-2 unit should be discarded at least every 24 hours.” Additionally, the label provides, “VIRAZOLE brand of Ribavirin is supplied as 6 grams of lyophilized powder per 100 mL vial for aerosol administration only. By sterile technique, reconstitute drug with a minimum of 75 mL of Sterile Water for Injection, USP or Inhalation in the original 100 mL glass vial. Shake well. Transfer to the clean, sterilized 500 mL SPAG-2 reservoir and further dilute to a final volume of 300 mL with Sterile Water for Injection, USP or Inhalation. The final concentration should be 20 mg/mL.” There is a chance that undesirable visual particles or discoloration may occur in the currently available reconstituted and diluted solution.

Reconstitution by sterile technique requires special skills to avoid microbial contamination. The chances of needle stick injury or exposure during reconstitution and dilution are high. Moreover, as Ribavirin is teratogenic, women in child bearing age group have to be extremely cautious in handling such product. Reconstitution and dilution up to 300 mL is required for administration of the currently marketed inhalation solution. There is risk of overdose, or under dose due to human error, if the dilution is not done properly. There is no stability data available for the reconstituted VIRAZOLE® solution in the labeling and the literature suggests that a 67 mg/mL solution is chemically stable only for 45 days in a syringe or glass vial. However, after reconstitution the product cannot be claimed as sterile due to possible direct and/or indirect exposure to environment.

Furthermore, the currently available VIRAZOLE® is prepared by a lyophilization process, which is a lengthy and complex process. Lyophilization is a longer process than filling a ready to use solution directly into a suitable container. The cost involved in lyophilization of a solution containing 6 gm Ribavirin is significantly higher than filling a simple liquid formulation into e.g. a bag. Moreover, lyophilized pharmaceutical products are always prepared by aseptic processes of sterilization, which are generally known to be a less reliable method of sterilization than terminal sterilization. Based on EMEA and FDA recommendations, terminally sterilized products are preferable to aseptic processing due to risks involved in aseptic processing.

FDA guidelines explain the basic differences between the production of sterile drug products using aseptic processing and production using terminal sterilization. Terminal sterilization usually involves filling and sealing product containers under high-quality environmental conditions. Products are filled and sealed in this type of environment to minimize exposure of the microbial and particulate contaminants to the in-process product and to help ensure that the subsequent sterilization process is successful. In most cases, the product, container, and closure have low bioburden, but they are not sterile. The product in its final container is then subjected to a sterilization process such as heat or irradiation.

In an aseptic processes, the drug product, container, and closure are first subjected to sterilization methods separately, as appropriate, and then brought together. Because there is no process to sterilize the product in its final container, it is critical that containers be filled and sealed in an extremely high-quality environment. Aseptic processing involves more variables than terminal sterilization. Before aseptic assembly into a final product, the individual parts of the final product are generally subjected to various sterilization processes. For example, glass containers are subjected to dry heat; rubber closures are subjected to moist heat; and liquid dosage forms are subjected to filtration. Each of these manufacturing processes requires validation and control. Each process can introduce an error that ultimately could lead to the distribution of a contaminated product. Any manual or mechanical manipulation of the sterilized drug, components, containers, or closures prior to or during aseptic assembly poses the risk of contamination and thus necessitates careful control.

A terminally sterilized drug product, on the other hand, undergoes final sterilization in a sealed container, thus limiting the possibility of error.

There have been recalls to the currently marketed VIRAZOLE® (Ribavirin) for Inhalation Solution, 6 gm in 100 mL vial lyophilized product. Valeant Pharmaceuticals North America LLC was issued a voluntarily recall of one lot of VIRAZOLE® (ribavirin powder for solution), 100 mL, 6 g Vial, 4-pack to the user level. A Jan. 2, 2015 post by FDA states that, “Inhalation of a non-sterile product with microbial contamination into the airways could increase the risk of respiratory infection. The risk is higher in patients who are immunocompromised (because of underlying disease), and are more susceptible.”

A Ribavirin Inhalation Solution prepared by terminal sterilization would provide a much higher level of sterility assurance for e.g. sick infants with respiratory syncytial virus (RSV) lung infection.

The NDA for VIRAZOLE® was approved in 1985. There has been a long-felt need for lower cost, generic alternatives to VIRAZOLE®.

There has been a long-felt, unmet need to have formulations of Ribavirin that can be terminally sterilized.

There has been a long-felt need for Ribavirin Inhalation formulations that are suitable for administration to infants that are sterilized by terminal sterilization.

There is a desire to have ready to use solution formulations of Ribavirin that do not have to be reconstituted and diluted prior to administration by inhalation.

There is a desire to have ready to use sterile solution formulations of Ribavirin that do not have to be reconstituted and diluted prior to administration.

There is a desire for formulations of Ribavirin for administration by inhalation that are not provided to health care providers as a lyophilized product that requires further reconstitution.

There is a desire for sterile, aqueous compositions of Ribavirin that are stable for an extended period of time.

There is a desire for stable aqueous solutions of Ribavirin that are free of preservatives.

There is a desire for stable aqueous solutions of Ribavirin that are free of microbial contaminants.

There is a desire for stable aqueous solutions of Ribavirin that are free of sugars and carbohydrates.

There is a desire for methods of treating respiratory syncytial virus in a patient in need thereof by pouring a ready to use 20 mg/mL Ribavirin solution directly into a SPAG-2 nebulizer reservoir and nebulizing the Ribavirin solution without further dilution thereof.

### SUMMARY OF THE INVENTION

The foregoing is achieved by the present invention, which provides stable, liquid, parenteral pharmaceutical compositions of Ribavirin that are ready to use and do not require reconstitution and/or dilution prior to administration. The invention further concerns methods of preparing such compositions by a sterilization process, preferably terminal sterilization.

One aspect of the invention is preparing Ribavirin Inhalation solutions packaged in a container, which do not require further reconstitution and/or dilution and potential exposure to health care workers.

In one aspect, the invention provides a method of manufacturing a stable, sterile, aqueous pharmaceutical composition of Ribavirin comprising the steps of solubilizing Ribavirin active ingredient in water, filling the Ribavirin solution into a primary packaging container, and terminally sterilizing the Ribavirin solution in the primary packaging container.

In some embodiments, the method comprises a step of filtering the Ribavirin solution through a filter, preferably a 0.22 μm filter, prior to the step of packaging the Ribavirin solution. In some of those methods, the filter is a PVDF filter.

In certain embodiments, the method comprises the step of sealing the primary packaging container before the step of terminally sterilizing.

In some of embodiments, the primary packaging is a vial and the step of sealing comprises placing a stopper into the vial. In some of those embodiments, the step of sealing further comprises placing a flip-off seal over the stopper.

In certain preferred embodiments, the terminal sterilization method is autoclaving at about 121° C. for about 10 to 15 min.

In some embodiments, the primary packaging container is a bag, bottle or vial. In some of those embodiments, the primary packaging is selected from polysulfone, polycarbonate, polypropylene, polyethylene (LDPE or HDPE), ethylene/propylene copolymers, polyolefins (eg. COC, COP and the like), acrylic-imide copolymers, polyester (e.g. PET, PEN and the like), Teflon, Nylon, acetal (Delrin), polymethylpentene, PVDC, ethylvinylacetate, AN-copolymer, CZ resin containers.

In certain embodiments, the method comprises a step of packaging the terminally sterilized product into secondary packaging. In certain of those embodiments, the secondary packaging is selected from the group consisting of aluminum pouch, plastic bag, and paper carton.

In some embodiments, the concentration of Ribavirin in the Ribavirin solution is 10 mg/mL to 40 mg/mL. In some preferred embodiments, the concentration of Ribavirin in the Ribavirin solution is 20 mg/mL.

The inventive methods produce Ribavirin solution that is stable in the primary packaging for an extended period of time at normal storage conditions. In certain embodiments, the Ribavirin solution is stable for 6 months at 40° C. In certain of those embodiments, the Ribavirin solution is stable for 6 months at 40° C. with 25% to 75% RH. In certain preferred embodiments, the Ribavirin solution is stable for 6 months at 40° C./75% RH.

In some embodiments, the Ribavirin solution is stable in the secondary packaging for 6 months or 12 months at 25° C. In some of those embodiments, the solution is stable at 25° C. with 25% to 60% RH. In some preferred embodiments, the Ribavirin solution is stable in the secondary packaging for 6 months or 12 months at 25° C./60% RH.

In another aspect, the invention comprises a sterile, ready to use pharmaceutical composition of Ribavirin comprising a primary packaging container containing a sterile Ribavirin solution having a concentration of 10 mg/mL to 40 mg/mL Ribavirin and a seal sealing the primary packaging container, wherein the Ribavirin solution is free of viable microbial contamination by after testing for sterility in accordance with 40 USP <71> using Trypticase Soy Broth and Fluid Thioglycollate Medium. In certain preferred embodiments, the concentration of Ribavirin is 20 mg/mL.

In some embodiments, the primary packaging container is a plastic or glass container.

In certain embodiments, the Ribavirin solution is sterilized by terminal sterilization. In some of those embodiments, the terminal sterilization is autoclaving at about 121° C. for about 10 to 15 min. In other embodiments, the Ribavirin solution is sterilized by gamma irradiation. In yet other embodiments, the Ribavirin solution is sterilized by sterile filtration.

The sterile compositions are suitable for administration via a parenteral route of administration, in particular, administration via Inhalation route of administration.

The sterile compositions are free of precipitation. In preferred embodiments, the sterile compositions are additionally free of flavoring, and preservatives. In some embodiments, the sterile compositions are free of sugars.

In yet another aspect, the invention provides a stable, liquid pharmaceutical composition of Ribavirin comprising a primary packaging container containing a Ribavirin solution consisting essentially of 10 mg/mL to 40 mg/mL Ribavirin and water and a seal sealing the primary packaging container, wherein the composition is sterilized by a sterilization process selected from terminal sterilization, gamma irradiation and sterile filtration. The product is stable in the primary packaging for an extended period of time at normal storage conditions.

In some embodiments, the composition is stable in the primary packaging for an extended period of time at normal storage conditions. In certain embodiments, the composition is stable for 6 months at 40° C. In certain of those embodiments, the composition is stable for 6 months at 40° C. with 25% to 75% RH. In certain preferred embodiments, the composition is stable for 6 months at 40° C./75% RH.

In some embodiments, the composition is stable in the secondary packaging for 6 or 12 months months at 25° C. In some of those embodiments, the composition is stable at 25° C. with 25% to 60% RH. In some preferred embodiments, the composition is stable in the secondary packaging for 6 months or 12 months at 25° C./60% RH.

The stable aqueous compositions are suitable for parenteral administration and are free of precipitates, flavoring, preservatives and sugars.

The invention also provides a method of treating respiratory syncytial virus in a patient in need thereof by pouring a 20 mg/mL Ribavirin solution into a SPAG-2 nebulizer reservoir and nebulizing the Ribavirin solution without further dilution thereof. In certain preferred embodiments, the Ribavirin solution consists essentially of Ribavirin and water. In some preferred embodiments, the Ribavirin solution consists of Ribavirin and water.

### DETAILED DESCRIPTION

The currently marketed formulation, VIRAZOLE®, requires reconstitution with 75 mL sterile water for injection and further diluted to 300 mL. Risk of exposure to Ribavirin is involved for nurses and other medical staff who perform reconstitution and dilution of Ribavirin. Ready to use Ribavirin Inhalation solutions of the present invention do not require reconstitution or further dilution, which are safer for medical staff by minimizing the time of handling and exposure.

Moreover, the currently approved VIRAZOLE® is a lyophilized product. The compositions of the present invention are prepared without lyophilization and are much simpler and cost-effective products.

The term “lyophilization” (also known as freeze-drying, lyophilisation, or cryodesiccation) means a process of removal water or other solvents by freezing a material containing water and/or other solvents followed by reducing the surrounding pressure to allow the frozen water and/or other solvents in the material to sublimate directly from the solid phase to the gas phase.

The formulations of the present invention comprise about 5 mg/mL to 125 mg/mL, preferably 10-40 mg/ml Ribavirin, most preferably about 20 mg/mL Ribavirin, or a pharmaceutically acceptable salt thereof, and water. In preferred embodiments, the Ribavirin is Ribavirin free base or a pharmaceutically acceptable salt thereof.

As used herein the term “Ribavirin” refers to the free base. The term “Ribavirin active ingredient” includes Ribavirin and pharmaceutically acceptable polymorphs, hydrates and salts thereof.

The term “pharmaceutically acceptable” as used herein means that which is useful in preparing a pharmaceutical composition that is generally safe and non-toxic.

The formulations comprise greater than about 88% water. In preferred embodiments, the formulations comprise greater than 90% water, more preferably 95% to 99% water. Most preferably, the formulations comprise 96% to 99% water. Preferably, the formulations comprise water for injection.

All % of solvents herein refer to volume %, unless otherwise specified. The term “% v/v” (also written as “v/v %”) means the volume of a solute in the total volume of solution. As one skilled in the art would understand, when the solute is a liquid, sometimes it is convenient to express its concentration in volume/volume percent. The calculation of “% v/v” is: Concentration solute (v/v %)=volume solute (mL) Total volume of solution (mL)×100.

As used herein, the term “about” is defined as ±10%, preferably ±5%.

In certain embodiments, the compositions of the present invention can be formulated as “ready to use” compositions, which refers to premixed compositions that are suitable for administration to a patient without dilution. For example, in certain embodiments, the compositions of the present invention are “ready to use” upon removing the compositions from a sealed container or vessel.

In certain embodiments, the compositions of the present invention can be formulated as a “single use dosage,” which refers to a premixed composition that is disposed within a sealed container or vessel as a one dose per container or vessel formulation.

As used herein, the term “seal” refers to any device or substance used to join two surfaces of a container or packaging so as to prevent them from coming apart or to prevent the contents from escaping. The step of “sealing” a container or packaging with a seal can occur prior to, simultaneous with, or after a Ribavirin solution is placed in the container or packaging. For instance, the invention is meant to include blow fill seal technologies in which a container is formed, filled, and sealed in a continuous process without human intervention, in a sterile enclosed area inside a machine. The invention can also include conventional processes in which a glass vial is sealed with a stopper. Other means of sealing and packaging pharmaceutical solutions in primary and secondary packaging are well known in the drug packaging arts and are meant to be included herein.

As used herein, the term “primary packaging” refers to materials that are in direct contact with the Ribavirin active ingredient. The primary packaging components (e.g. bag, bottles, vials, closures) are in direct physical contact with the product whereas the secondary components are not.

As used herein, the term “secondary packaging” refers to packaging that is not in direct contact with the Ribavirin active ingredient. Secondary packaging may include pouches, cartons or bags, such as aluminum pouches, paper cartons and plastic bags. Often, the secondary packaging addresses patient adherence and provides information on how to deliver and use a pharmaceutical product, such as labeling.

Guidelines on suitable packaging for pharmaceutical products are published by e.g. the World Health Organization and are well known to those of skill in the art.

The term “therapeutically effective amount” as used herein means that the amount of Ribavirin contained in the composition administered is of sufficient quantity to achieve the intended purpose, such as, in this case treatment of RSV.

The formulations of the invention may also comprise suitable excipients.

Suitable excipients include but are not limited to suitable pharmaceutical buffer, pH adjusting agent, anti-oxidant, preservative, chelating agent, tonicity adjusting agent or a combination of thereof. These excipients can be used in proportion of about 0-20% of the formulation individually, or in the form of any combination, more preferably about 5%.

pH regulating agents include buffers such as acetate, citrate, phosphate, borate, carbonate etc., sodium hydroxide, hydrochloric acid etc.

The formulation is stable at about pH 3.5 to pH 7.0. More preferably, the formulation has a pH of about pH 4.0 to 6.5.

The formulations can be directly prepared in or transferred to a suitable container. In some embodiments, the primary packaging container is a bag, bottle or vial. In certain configurations, the bag or vial may be made of polymeric materials, such as but not limited to, polypropylene, ethylvinyl acetate, polyethylene, polyvinylchloride, cyclic olefin polymer and glass materials and polysulfone, polycarbonate, polypropylene, polyethylene (LDPE or HDPE), polymethylpentene, ethylene/propylene copolymers, polyolefins, acrylic-imide copolymers, Teflon, Nylon, acetal (Delrin), PVDC, ethylvinylacetate, AN-copolymer etc. In one exemplary embodiment, the container is plastic bag. In addition to plastic bags, polyester (e.g. PET, PEN and the like), CZ resin containers, polypropylene and similar resins can be used as rigid containers.

A preferred plastic bag is selected from but not limited to the group of GALAXY®, INTRAVIA®, SOLOMIX®, STEDIM® 71, STEDIM® 100, VIAFLEX®, EXCEL®, VISIV®, VIAFLO™, ADDEASE®, ADD-VANTAGE®, DUPLEX™, FIRST CHOICE™, PROPYFLEX™ and BFS™. Material of construction of the bag can be selected from but not limited to the group of polypropylene, polyethylene, polyvinyl chloride, ethylenvinyl acetate, Bis(2-ethylhexyl) phthalate, polyolefin, polysulfone, polycarbonate, ethylene/propylene copolymers, acrylic-imide copolymers, polyester, Teflon, Nylon, acetal (Delrin), polymethylpentene, PVDC, ethylvinylacetate, or combination thereof.

Examples of other types of suitable containers include glass vials or bottles, plastic vials or bottles or any type of container which can hold desired volume of Ribavirin solution in sterile condition.

The formulations of the present invention may be used for parenteral route, preferably for inhalation route of administration, by the use of any device or apparatus.

In preferred embodiments, the inventions are sterile. As used here in the term “sterile” means a product which meets the criteria of sterility according to the US Pharmacopoeia 40-NF 35<71> (“40 USP”), and which provides a therapeutically acceptable Ribavirin composition. Further regulations for sterility of the final product include the European Pharmacopoeia (Ph. Eur. section 2.6.1), and the Japanese Pharmacopoeia (JP section 54). These methods have been harmonized with the USP methods and results generated under these sources can be considered equivalent to testing conducted according to USP <71>. Preferably, the therapeutically acceptable Ribavirin compositions have been produced by a method which provides assurance of sterility according to the US Pharmacopoeia 40-NF 35<71> or USP 41<71>. Sterility testing of the inventive compositions has been conducted in accordance with both versions of USP and there are no changes to this chapter from 40 USP to 41 USP.

Sterile solutions are considered free of viable microbial contamination. In contrast, prior oral solutions are not labeled as sterile and cannot be considered as free of viable microbial contamination. It is known in the art that ingredients use to formulate oral solutions of Ribavirin are susceptible to at least trace amounts of microbial contamination and will promote the growth of viable microorganisms.

In some embodiments, the Ribavirin solutions of the invention may be filtered prior to filling in primary packaging. Suitable filters include, but are not limited to, those constructed of PVDF, PES, PTFE, Nylon and cellulose. Filters of various size are known in the art. In some embodiments, Ribavirin solution may be filtered through a 0.22 or 0.45 micron sterilizing filter. In certain preferred embodiments, Ribavirin solution is filtered through a 0.22 micron PVDF filter.

In preferred embodiments, the primary packaging container is placed in secondary packaging that comprises aluminum laminated pouches. The secondary packaging may include single use or multi use containers. In some preferred embodiments, the secondary packaging contains a single use container and labeling. In other preferred embodiments, the secondary packaging contains several primary packaging containers and labeling.

The Ribavirin in the solutions according to the invention will essentially maintain the same pharmacological activity and physico-chemical properties/its chemical purity and physical form as the starting material from which it is prepared, i.e. the degradation, and especially the chemical degradation, caused by any sterilization process will be limited.

As used herein, the term “sterilization” refers to a means for the control of microbial population. The term “sterilization process” refers to a process that either kills or removes viable microorganisms.

The sterile compositions may be subjected to a sterilization process. Sterilization may be performed by any of the means commonly known to those of skill in the art and discussed in the current version of USP <71>, or 41 USP <1211>, and the Second Supplement to USP 41-NF36, the contents of which are incorporated herein. In some embodiments, sterilization is performed by a terminal sterilization method, such as moist heat or dry heat sterilization. In other embodiments, sterilization is performed by gamma irradiation or sterile filtration. In certain preferred embodiments, sterile product is produced by terminally sterilizing a Ribavirin solution in an autoclave at about 121° C. for about 10 to 15 min. As used herein, use of an autoclave or “autoclaving” is considered a method of moist heat sterilization. The aforementioned sterilization methods are known to kill and/or remove any viable microorganism contained in a formulation and are effective in producing compositions that are free of viable microbial contamination and/or will have zero positive samples when tested in accordance with the sterility testing methods in USP <71>.

The compositions disclosed herein are stable for an extended period of time at normal storage conditions. As used herein, “stable” is defined as no more than about a 10% loss of Ribavirin under typical commercial storage conditions. Preferably, formulations of the present inventions will have no more than about a 10% loss of Ribavirin, more preferably, no more than about a 5% loss of Ribavirin, under typical commercial storage conditions.

As used herein, an “extended period of time” means 3 months or greater.

Storage conditions refers to those long term, intermediate and accelerated conditions discussed in ICH guidelines for Stability Testing of New Drug Substances and Products Q1A(R2), Step 4 version dated 6 Feb. 2003, the contents of which are incorporated herein by reference. Namely, storage conditions include 5° C.±3° C., 25° C.±2° C./60% RH±5% RH, 30° C.±2° C./65% RH±5% RH, and 40° C.±2° C./75% RH±5% RH. As used herein, storage of compositions refers to storage within a container closure system. For instance, accelerated storage conditions for semi-permeable containers, such as polypropylene bag, should not exceed 40° C./25% RH. Storage conditions can also refer to ambient conditions.

The term “ambient” as used herein refers to uncontrolled atmospheric conditions in the room or place. For purposes of experiments conducted by the inventors, ambient laboratory conditions aimed to achieve 25° C.±2° C./60% RH±5% RH. However, such conditions were not strictly maintained and monitored in the ambient environment.

Analysis of the liquid formulations of the present invention can be performed using techniques known in the art, including, for example, HPLC, gas chromatography, and NMR. After exposure to typical commercial storage conditions, analysis of the formulations of the present invention will indicate that the formulation contains no less than about 90% of the amount of Ribavirin present prior to exposure to the storage conditions. Preferably, analysis will indicate that the formulation contains no less than about 95% of the amount of Ribavirin present prior to exposure to the storage conditions. More preferably, analysis will indicate that the formulation contains no less than about 98% of the amount of Ribavirin prior to exposure to the storage conditions.

In preferred embodiments of the present invention, analysis of the formulations of the present invention will indicate that the formulation contains no less than about 90% of the amount of Ribavirin present prior to exposure to storage conditions that include temperatures of about 25° C. and time periods of about 30 days (about 1 month) to about 365 days (about 1 year). Preferably, analysis of the formulations of the present invention will indicate that the formulation contains no less than about 90% of the amount of Ribavirin present prior to exposure to storage conditions that include temperatures of about 25° C. and time periods of about 30 days (about 1 month), about 90 days (about 3 months), about 180 days (about 6 months), and about 365 days (12 months). Preferably, analysis will indicate that the formulation contains no less than about 95% of the amount of Ribavirin present prior to exposure to storage conditions that include temperatures of about 25° C. and time periods of about 30 days (about 1 month) to about 365 days (about 1 year). More preferably, analysis will indicate that the formulation contains no less than about 95% of the amount of Ribavirin present prior to exposure to storage conditions that include temperatures of about 25° C. and time periods of about 30 days (about 1 month), about 90 days (about 3 months), 180 days (about 6 months), and about 365 days (about 12 months).

In preferred embodiments of the present invention, analysis of the formulations of the present invention will indicate that the formulation contains no less than about 90% of the amount of Ribavirin present prior to exposure to storage conditions that include temperatures of about 40° C. and time periods of about 30 days (about 1 month) to about 365 days (about 1 year). Preferably, analysis of the formulations of the present invention will indicate that the formulation contains no less than about 90% of the amount of Ribavirin present prior to exposure to storage conditions that include temperatures of about 40° C. and time periods of about 30 days (about 1 month), about 90 days (about 3 months), and about 180 days (about 6 months). Preferably, analysis will indicate that the formulation contains no less than about 95% of the amount of Ribavirin present prior to exposure to storage conditions that include temperatures of about 40° C. and time periods of about 30 days (about 1 month) to about 365 days (about 1 year). More preferably, analysis will indicate that the formulation contains no less than about 95% of the amount of Ribavirin present prior to exposure to storage conditions that include temperatures of about 40° C. and time periods of about 30 days (about 1 month), about 90 days (about 3 months), about 180 days (about 6 months), about 240 days (about 9 months), and about 365 days (about 1 year).

In preferred embodiments, the amount of triazole carboxylic acid, related compound ‘A’ (1-beta-Dribofuranosyl-1H-1,2,4-triazole-3-carboxylic acid), related compound ‘D’ (1H-1,2,4-triazole-3-carboxamide) and related compound ‘B’ (1-α-D-ribofuranosyl-1H-1,2,4-triazole-3-carboxamide (α-anomer)) is present in the inventive compositions at not more than 0.25% individually.

In some preferred embodiments, any individual unspecified impurity is present in the compositions at not more than 0.10%.

The invention is illustrated but not limited by the following examples.

### EXAMPLES

### Example 1. Composition of Ribavirin Inhalation Solution, 10 mg/mL

| Sr. No. | Ingredient | Quantity | (%) |
|---|---|---|---|
| 1 | Ribavirin | 10 mg | 1% w/v |
| 2 | Water | q.s. to 1 mL | q.s. to 100% |

### Example 2. Composition of Ribavirin Inhalation Solution, 20 mg/mL

| Sr. No. | Ingredient | Quantity | (%) |
|---|---|---|---|
| 1 | Ribavirin | 20 mg | 2% w/v |
| 2 | Water | q.s. to 1 mL | q.s. to 100% |

### Example 3. Composition of Ribavirin Inhalation Solution, 40 mg/mL

| Sr. No. | Ingredient | Quantity | (%) |
|---|---|---|---|
| 1 | Ribavirin | 40 mg | 4% w/v |
| 2 | Water | q.s. to 1 mL | q.s. to 100% |

### Example 4. Method of Preparation

Take partial quantity of water. Add and dissolve required quantity of Ribavirin. Make up the volume up to batch size. Filter the solution through 0.22μ filter. Fill desired volume in plastic bag. Optionally autoclave at about 121° C. for about 10 to 15 min. Pack in suitable secondary packaging.

### Example 5: Method of Preparation

Approximately 1800 mL of water for injection was collected in glass beaker. Required quantity of Ribavirin was added in collected water for injection and mixed to get clear colorless solution. Volume of the batch was made up to 2.0 liter using water for injection. Prepared bulk solution was filtered through 0.22μ PVDF filter. Filtered bulk solution was filled in polypropylene bag and stoppered with twist-off port. These bags are further packed in aluminum pouches and stored on stability.

Stability data of a 20 mg/mL formulation is given in Example 9.

### Example 6

Filtered bulk solution of Example 5 was filled in USP type I glass vials and stoppered with bromobutyl stopper. The vials were sealed with aluminum flip-off seals. To study the thermal effect on solution, the sealed vial was autoclaved at 121° C. for 15 min.

### Example 7

Filtered bulk solution of Example 5 was also filled in polypropylene bags and the bags were autoclaved at 121° C. for 15 min for terminal sterilization. Terminally sterilized bag was tested for assay, related compounds and sterility test. For sterility test, sample was incubated for 14 days in Trypticase Soy Broth and Fluid Thioglycollate Medium, the product was found to be sterile as below:

| Number Tested | | Number Positive | |
|---|---|---|---|
| Trypticase | Fluid | Trypticase | Fluid |
| Soy | Thioglycollate | Soy | Thioglycollate |
| Broth | Medium | Broth | Medium |
| 1 | 1 | 0 | 0 |

For chemical testing, initial results of autoclaved sample are described in below table:

| Test | Result |
|---|---|
| Assay | 100.30% |
| Triazole Carboxylic Acid | <LOQ (0.03%) |
| Related Compound ‘A’ | 0.07% |
| Related Compound ‘D’ | 0.05% |
| Related Compound ‘B’ | <LOQ (0.03%) |
| Any Unknown Impurity | <LOQ (0.03%) |
| Total Impurities | 0.12% |

Related Compound ‘A’: 1-beta-Dribofuranosyl-1H-1,2,4-triazole-3-carboxylic acid; Related Compound 1H-1,2,4-triazole-3-carboxamide, Related Compound ‘B’: 1-α-D-ribofuranosyl-1H-1,2,4-triazole-3-carboxamide (α-anomer)

HPLC Method for Related Compounds of Ribavirin in Ribavirin Inhalation solution, 20 mg/mL using HPLC with UV detector:
a. Dilution of Sample: Dilute the sample to produce final concentration of 1 mg/mL of Ribavirin using water
b. Column: C-18, 4.6×250 mm, 3.5μ (Waters X Select HSS T3 or equivalent)
c. Mobile phase A for gradient program: Aqueous phosphate buffer pH 4
d. Mobile phase B for gradient program: Aqueous phosphate buffer pH 4: Acetonitrile (90:10)
e. Wavelength: 207 nm
f. Flow rate: 0.8 mL/min
g. Gradient Program:

| Time | Mobile phase A | Mobile phase B |
|---|---|---|
| (min) | (%) | (%) |
| 0.01 | 100 | 0 |
| 5.0 | 100 | 0 |
| 12.0 | 15 | 85 |
| 15.0 | 0 | 100 |
| 20.0 | 0 | 100 |
| 22.0 | 100 | 0 |
| 32.0 | 100 | 0 |

Method for Assay of Ribavirin in Ribavirin Inhalation solution, 20 mg/mL using HPLC with UV detector:
a. Dilution of Sample: Dilute the sample to produce final concentration of 1 mg/mL of Ribavirin using water
b. Column: C-18, 4.6×250 mm, 3.5μ (Waters X Select HSS T3 or equivalent)
c. Mobile phase for Isocratic program: Aqueous phosphate buffer pH 4: Acetonitrile (95:05)
d. Wavelength: 207 nm
e. Flow rate: 0.8 mL/min

### Example 8: Sterility of Solutions Upon Extended Storage

The sterility of Ribavirin Inhalation Solution (20 mg/mL) filled in polypropylene bag (supplied by Technoflex) as primary packaging, subsequently wrapped with laminated aluminum pouch and stored for 9 months at 25° C./60% RH was tested for sterility test by Membrane Filtration in accordance with current USP <71>. The samples passed the USP sterility test.

### Example 9

Stability Data and initial sterility data is provided in Tables 1-5.

The formulations of present invention may be directly administered by continuous aerosol administration for 12-18 hours per day for 3 to 7 days. The inventive methods and compositions are able to provide the recommended drug concentration of 20 mg/mL. Using such concentration the average aerosol concentration for a 12 hour delivery period would be 190 micrograms/liter of air. It is advantageous that the inventive compositions can be used to treat respiratory syncytial virus in a patient in need thereof by directly pouring a 20 mg/mL Ribavirin solution into a SPAG-2 nebulizer reservoir and nebulizing the Ribavirin solution without further dilution thereof.

A method of treating respiratory syncytial virus in a patient in need thereof comprises removing a primary packaging container having a 20 mg/mL Ribavirin solution from secondary packaging, pouring the 20 mg/mL Ribavirin solution into a SPAG-2 nebulizer reservoir and nebulizing the Ribavirin solution without further dilution thereof. In certain preferred embodiments, the Ribavirin solution consists essentially of Ribavirin and water. In some preferred embodiments, the Ribavirin solution consists of Ribavirin and water

The prior art inhalation solutions require that lyophile is first diluted to a concentration of 60 mg/mL in a flask then further diluted in the SPAG-II nebulizer to a concentration of 20 mg/mL. The invention saves time for healthcare workers by eliminating dilution steps and errors that can be caused by the dilution step. The invention is further advantageous in that it minimizes exposure of healthcare workers to the teratogenic lyophilized Ribavirin active ingredient, and a higher 60 mg/mL concentrated solution of Ribavirin during dilution of the lyophile.

It is envisioned that the pharmaceutical formulations and preparations of the present invention can be administered in combination with other agents where the other agents are given prior to, or subsequent to the administration of the formulations or preparations of the present invention. Pharmaceutically acceptable agents are known in the art.

It should be noted that the invention in its broader aspects is not limited to the specific details, representative compositions, methods, and processes, and illustrative examples described in connection with the preferred embodiments and preferred methods. Modifications and equivalents will be apparent to practitioners skilled in this art and are encompassed within the spirit and scope of the appended claims

TABLE 1
Finished Product test result of Ribavirin
Inhalation solution 20 mg/mL
(terminally sterilized at 121° C. for 15 min in vial)

| Test | Result |
|---|---|
| Description | Clear, colorless solution |
| Assay | 102.1% |
| Triazole Carboxylic Acid | <LOQ (0.03%) |
| Related Compound ‘A’ | 0.09% |
| Related Compound ‘D’ | 0.09% |
| Related Compound ‘B’ | <LOQ (0.03%) |
| Any Unknown Impurity | <LOQ (0.03%) |
| Total Impurities | 0.18% |

TABLE 2
Stability study of Ribavirin Inhalation solution 20 mg/mL (Product without
terminal sterilization, packed in PP bag with aluminum laminated pouch)

| | | | | | Triazole | Related | Related | Related | Any | |
|---|---|---|---|---|---|---|---|---|---|---|
| Stability/storage | | | | | Carboxylic | Compound | Compound | Compound | Unknown | Total |
| condition condition | | | Description | Assay | Acid | ‘A’ | ‘D’ | ‘B’ | Impurity | Impurities |
| Initial | | | Clear, colorless | 102.7% | <LOQ (0.03%) | 0.06% | <LOQ (0.03%) | <LOQ (0.03%) | <LOQ (0.03%) | 0.06% |
| | | | solution | | | | | | | |
| 2-8° C. | 1 | Month | Clear, colorless | 103.2% | <LOQ (0.03%) | 0.06% | <LOQ (0.03%) | <LOQ (0.03%) | <LOQ (0.03%) | 0.06% |
| | | | solution | | | | | | | |
| | 2 | Months | Clear, colorless | 105.3% | <LOQ (0.03%) | 0.06% | <LOQ (0.03%) | <LOQ (0.03%) | <LOQ (0.03%) | 0.06% |
| | | | solution | | | | | | | |
| | 3 | Months | Clear, colorless | 100.2% | <LOQ (0.03%) | 0.07% | <LOQ (0.03%) | <LOQ (0.03%) | <LOQ (0.03%) | 0.07% |
| | | | solution | | | | | | | |
| 25° C./ | 1 | Months | Clear, colorless | 103.1% | <LOQ (0.03%) | 0.06% | <LOQ (0.03%) | <LOQ (0.03%) | <LOQ (0.03%) | 0.06% |
| 60% RH | | | solution | | | | | | | |
| | 2 | Months | Clear, colorless | 105.6% | <LOQ (0.03%) | 0.07% | <LOQ (0.03%) | <LOQ (0.03%) | <LOQ (0.03%) | 0.07% |
| | | | solution | | | | | | | |
| | 3 | Months | Clear, colorless | 102.7% | <LOQ (0.03%) | 0.07% | <LOQ (0.03%) | <LOQ (0.03%) | <LOQ (0.03%) | 0.07% |
| | | | solution | | | | | | | |
| | 6 | Months | Clear, colorless | 106.5% | <LOQ (0.03%) | 0.09% | <LOQ (0.03%) | <LOQ (0.03%) | <LOQ (0.03%) | 0.09% |
| | | | solution | | | | | | | |
| | 15 | Months | Clear, colorless | 102.8% | <LOQ (0.03%) | 0.10% | <LOQ (0.03%) | <LOQ (0.03%) | <LOQ (0.03%) | 0.10% |
| | | | solution | | | | | | | |
| 40° C./ | 1 | Month | Clear, colorless | 102.3% | <LOQ (0.03%) | 0.08% | <LOQ (0.03%) | <LOQ (0.03%) | <LOQ (0.03%) | 0.08% |
| 75% RH | | | solution | | | | | | | |
| | 2 | Months | Clear, colorless | 104.9% | <LOQ (0.03%) | 0.11% | <LOQ (0.03%) | <LOQ (0.03%) | <LOQ (0.03%) | 0.11% |
| | | | solution | | | | | | | |
| | 3 | Months | Clear, colorless | 101.3% | <LOQ (0.03%) | 0.15% | <LOQ (0.03%) | <LOQ (0.03%) | <LOQ (0.03%) | 0.15% |
| | | | solution | | | | | | | |
| | 6 | Months | Clear, colorless | 105.3% | <LOQ (0.03%) | 0.22% | <LOQ (0.03%) | <LOQ (0.03%) | <LOQ (0.03%) | 0.22% |
| | | | solution | | | | | | | |
| 50° C. | 1 | Month | Clear, colorless | 102.3% | <LOQ (0.03%) | 0.11% | <LOQ (0.03%) | <LOQ (0.03%) | <LOQ (0.03%) | 0.12% |
| | | | solution | | | | | | | |

TABLE 3
Stability study of Ribavirin Inhalation solution 20 mg/mL (Product exposed
to 121° C. for 15 min and packed in PP bags with aluminum laminated pouch)

| | | | | | Triazole | Related | Related | Related | Any | |
|---|---|---|---|---|---|---|---|---|---|---|
| Stability/storage | | | | | Carboxylic | Compound | Compound | Compound | Unknown | Total |
| condition condition | | | Description | Assay | Acid | ‘A’ | ‘D’ | ‘B’ | Impurity | Impurities |
| Initial | | | Clear, colorless | 102.3% | <LOQ (0.03%) | 0.10% | 0.11% | <LOQ (0.03%) | <LOQ (0.03%) | 0.21% |
| | | | solution | | | | | | | |
| 25° C./ | 1 | Months | Clear, colorless | 100.1% | <LOQ (0.03%) | 0.10% | 0.11% | <LOQ (0.03%) | <LOQ (0.03%) | 0.21% |
| 60% RH | | | solution | | | | | | | |
| | 2 | Months | Clear, colorless | 100.8% | <LOQ (0.03%) | 0.10% | 0.11% | <LOQ (0.03%) | <LOQ (0.03%) | 0.21% |
| | | | solution | | | | | | | |
| | 3 | Months | Clear, colorless | 100.3% | <LOQ (0.03%) | 0.11% | 0.12% | <LOQ (0.03%) | <LOQ (0.03%) | 0.23% |
| | | | solution | | | | | | | |
| | 6 | Months | Clear, colorless | 99.6% | <LOQ (0.03%) | 0.11% | 0.11% | <LOQ (0.03%) | <LOQ (0.03%) | 0.22% |
| | | | solution | | | | | | | |
| | 12 | Months | Clear, colorless | 99.8% | <LOQ (0.03%) | 0.13% | 0.11% | <LOQ (0.03%) | <LOQ (0.03%) | 0.24% |
| | | | solution | | | | | | | |
| 40° C./ | 1 | Month | Clear, colorless | 100.1% | <LOQ (0.03%) | 0.12% | 0.11% | <LOQ (0.03%) | <LOQ (0.03%) | 0.23% |
| 75% RH | | | solution | | | | | | | |
| | 2 | Months | Clear, colorless | 101.1% | <LOQ (0.03%) | 0.14% | 0.12% | <LOQ (0.03%) | <LOQ (0.03%) | 0.26% |
| | | | solution | | | | | | | |
| | 3 | Months | Clear, colorless | 100.0% | <LOQ (0.03%) | 0.17% | 0.13% | <LOQ (0.03%) | <LOQ (0.03%) | 0.30% |
| | | | solution | | | | | | | |
| | 6 | Months | Clear, colorless | 100.3% | <LOQ (0.03%) | 0.20% | 0.13% | <LOQ (0.03%) | <LOQ (0.03%) | 0.33% |
| | | | solution | | | | | | | |

TABLE 4
Stability study of Ribavirin Inhalation solution 20 mg/mL (Product exposed to
121° C. for 15 min and packed in PP bags without aluminum laminated pouch)

| | | | | Triazole | Related | Related | Related | Any | |
|---|---|---|---|---|---|---|---|---|---|
| Stability/storage | | | | Carboxylic | Compound | Compound | Compound | Unknown | Total |
| condition condition | | Description | Assay | Acid | ‘A’ | ‘D’ | ‘B’ | Impurity | Impurities |
| Initial | | Clear, colorless | 100.3% | <LOQ (0.03%) | 0.07% | 0.05% | <LOQ (0.03%) | <LOQ (0.03%) | 0.12% |
| | | solution | | | | | | | |
| | 1 Month | Clear, colorless | 101.8% | <LOQ (0.03%) | 0.12% | 0.11% | <LOQ (0.03%) | <LOQ (0.03%) | 0.23% |
| | | solution | | | | | | | |
| 40° C./ | 2 Months | Clear, colorless | 101.8% | <LOQ (0.03%) | 0.14% | 0.11% | <LOQ (0.03%) | <LOQ (0.03%) | 0.25% |
| 75% RH | | solution | | | | | | | |
| | 3 Months | Clear, colorless | 103.2% | <LOQ (0.03%) | 0.17% | 0.13% | <LOQ (0.03%) | <LOQ (0.03%) | 0.30% |
| | | solution | | | | | | | |
| | 6 Months | Clear, colorless | 104.8% | <LOQ (0.03%) | 0.23% | 0.14% | <LOQ (0.03%) | <LOQ (0.03%) | 0.37% |
| | | solution | | | | | | | |

TABLE 5
Stability study of Ribavirin Inhalation solution 20 mg/mL (Product filled in PP bag,
terminally sterilized at 121° C. for 15 min and packed in aluminum laminated pouch)

| | | | | Triazole | Related | Related | Related | Any | |
|---|---|---|---|---|---|---|---|---|---|
| Stability/storage | | | | Carboxylic | Compound | Compound | Compound | Unknown | Total |
| condition | | Description | Assay | Acid | ‘A’ | ‘D’ | ‘B’ | Impurity | Impurities |
| Initial | | Clear, colorless | 102.0% | <LOQ (0.03%) | 0.06% | 0.05% | <LOQ (0.03%) | <LOQ (0.03%) | 0.11% |
| | | solution | | | | | | | |
| | 1 Months | Clear, colorless | 102.5% | <LOQ (0.03%) | 0.07% | 0.05% | <LOQ (0.03%) | 0.03% (RRT 1.40) | 0.15% |
| | | solution | | | | | | | |
| 25° C./ | 2 Months | Clear, colorless | 102.0% | <LOQ (0.03%) | 0.09% | 0.08% | <LOQ (0.03%) | <LOQ (0.03%) | 0.17% |
| 60% RH | | solution | | | | | | | |
| | 3 Months | Clear, colorless | 103.5% | <LOQ (0.03%) | 0.07% | 0.05% | <LOQ (0.03%) | <LOQ (0.03%) | 0.12% |
| | | solution | | | | | | | |
| | 6 Months | Clear, colorless | 102.3% | <LOQ (0.03%) | 0.08% | 0.06% | <LOQ (0.03%) | <LOQ (0.03%) | 0.14% |
| | | solution | | | | | | | |
| | 1 Month | Clear, colorless | 102.1% | <LOQ (0.03%) | 0.09% | 0.06% | <LOQ (0.03%) | <LOQ (0.03%) | 0.15% |
| | | solution | | | | | | | |
| 40° C./ | 2 Months | Clear, colorless | 102.6% | <LOQ (0.03%) | 0.11% | 0.06% | <LOQ (0.03%) | <LOQ (0.03%) | 0.17% |
| 75% RH | | solution | | | | | | | |
| | 3 Months | Clear, colorless | 104.1% | <LOQ (0.03%) | 0.13% | 0.07% | <LOQ (0.03%) | <LOQ (0.03%) | 0.20% |
| | | solution | | | | | | | |
| | 6 Months | Clear, colorless | 102.8% | <LOQ (0.03%) | 0.19% | 0.07% | <LOQ (0.03%) | <LOQ (0.03%) | 0.26% |
| | | solution | | | | | | | |

What is claimed is:

1. A sterile, ready to use pharmaceutical composition of Ribavirin comprising
a primary packaging container containing a sterile aqueous Ribavirin solution having a concentration of 10 mg/mL to 40 mg/mL Ribavirin; and
a seal sealing the primary packaging container;
wherein the Ribavirin solution is free of viable microbial contamination in in accordance with a Sterility test method in 40 USP <71>.

2. The composition of claim 1, wherein the concentration of Ribavirin is 20 mg/mL.

3. The composition of claim 1, wherein the primary packaging container is a plastic or glass container.

4. The composition of claim 1, wherein the Ribavirin solution consists essentially of Ribavirin and water.

5. The composition of claim 4, wherein the Ribavirin solution consists of Ribavirin and water.

6. The composition of claim 1, wherein the Ribavirin solution is sterilized by terminal sterilization.

7. The composition of claim 6, wherein the terminal sterilization is autoclaving at about 121° C. for about 10 to 15 min.

8. The composition of claim 1, wherein the composition is suitable for administration via a parenteral route of administration.

9. The composition of claim 10, wherein the composition is suitable for administration via Inhalation route of administration.

10. A stable, liquid pharmaceutical composition of Ribavirin comprising
a primary packaging container containing a Ribavirin solution consisting essentially of 10 mg/mL to 40 mg/mL Ribavirin and water; and
a seal sealing the primary packaging container,
wherein the composition is sterilized by a sterilization process selected from terminal sterilization, gamma irradiation and sterile filtration.

11. The composition of claim 10, wherein the product is stable in the primary packaging for an extended period of time at normal storage conditions.

12. The composition of claim 10, wherein the product is stable in the secondary packaging for an extended period of time at normal storage conditions.

13. A method of manufacturing a stable, sterile, aqueous pharmaceutical composition of Ribavirin comprising the steps of:
solubilizing Ribavirin active ingredient in water;
filling the Ribavirin solution into a primary packaging container; and
terminally sterilizing the Ribavirin solution in the primary packaging container.

14. The method of claim 13, wherein the concentration of Ribavirin in the Ribavirin solution is 10 mg/mL to 40 mg/mL.

15. The method of claim 14, wherein the concentration of Ribavirin in the Ribavirin solution is 20 mg/mL.

16. The method of claim 13, further comprising the step of filtering the Ribavirin solution through a filter prior to the step of filling the Ribavirin solution.

17. The method of claim 16, wherein the filter is a sterilizing filter.

18. The method of claim 13, further comprising the step of sealing the primary packaging container before the step of terminally sterilizing.

19. The method of claim 13 wherein the primary packaging is a vial and the step of sealing comprises placing a stopper into the vial.

20. The method of claim 19, wherein the step of sealing further comprises placing a flip-off seal over the stopper.

21. The method of claim 13, where in the terminal sterilization method is autoclaving at about 121° C. for about 10 to 15 min.

22. The method of claim 13, wherein the primary packaging container is a bag, bottle or vial.

23. The method of claim 13, further comprising the step of packaging the terminally sterilized product into secondary packaging.

24. The method of claim 23, wherein the secondary packaging is selected from the group consisting of pouch, carton and bag.

25. The method of claim 13, wherein the Ribavirin solution is stable in the primary packaging for an extended period of time at normal storage conditions.

26. The method of claim 1, wherein the Ribavirin solution is stable in the secondary packing for an extended period of time at normal storage conditions.

27. A method of treating respiratory syncytial virus in a patient in need thereof by pouring a 20 mg/mL ready to use Ribavirin solution into a SPAG-2 nebulizer reservoir and nebulizing the Ribavirin solution without further dilution thereof.
